(19) United States
(12) Patent Application Publication
(10) Pub. No.: US 2011/0065212 A1
(43) Pub. Date: Mar. 17, 2011

(54) GRAFT POLYMER-CONTAINING SUBSTRATE, METHOD FOR PRODUCING THE SAME, TARGET SUBSTANCE-DETECTING ELEMENT, AND TARGET SUBSTANCE DETECTION KIT

(75) Inventors: Kazuhiro Ban, Tokyo (JP); Masato Minami, Kawasaki-shi (JP); Mayuko Kishi, Machida-shi (JP)

(73) Assignee: CANON KABUSHIKI KAISHA, Tokyo (JP)

(21) Appl. No.: 12/993,390

(22) PCT Filed: May 29, 2009
(86) PCT No.: PCT/JP2009/060299

§ 371 Date: Nov. 18, 2010

(30) Foreign Application Priority Data
May 30, 2008 (JP) 2008-143457

(51) Int. Cl. G01N 33/53; C08L 33/08

(52) U.S. Cl. 436/501; 524/502

(57) ABSTRACT

There is provided a graft polymer-containing substrate that reduces nonspecific adsorption of impurities onto a substrate surface and specifically captures a target substance. There is provided a graft polymer-containing substrate with a graft polymer formed on the surface thereof, characterized in that the graft polymer includes monomer units represented by the following general formulas (1) and (2):

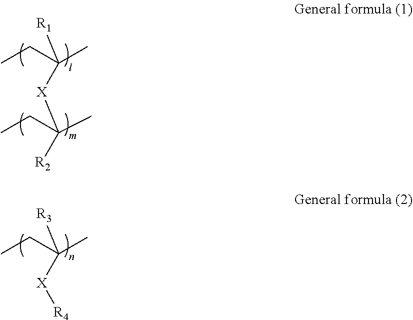

wherein R₁, R₂, and R₃ represent one of a hydrogen atom and a methyl group, R₄ represents a group including any of a carboxyl group, a succinimide group, an aldehyde group, an epoxy group, a bromo group, an amino group, and a maleimide group, X represents a hydrophilic crosslinking spacer having 15 or more atoms, and l, m, and n are an integer of 1 or greater.

### TECHNICAL FIELD

The present invention relates to a graft polymer-containing substrate, a method for producing the same, a target substance-detecting element, and a target substance detection kit. In particular, the present invention relates to a graft polymer-containing substrate including a target substance-capturing body that captures a target substance to a polymer membrane including a graft polymer and a biosensor for measuring a target substance that interacts with the target substance-capturing body with high sensitivity.

### BACKGROUND ART

Conventionally, intermolecular interactions have been utilized as means for detecting a target substance in a specimen. As a method utilizing interactions, it is common to allow a reaction by bringing one molecule immobilized on a substrate surface as a target substance-capturing body into contact with a specimen containing the target substance.

When the target substance that interacts with the immobilized target substance-capturing body is quantitatively measured, not only the target substance that interacts with a biomolecule but also substances adsorbed onto the substrate surface by nonspecific binding may be detected at the same time depending on the properties of the substrate surface or the immobilization method, which results in deterioration of the minimum detection sensitivity in a sensor that requires detection of trace amounts. Therefore, a technique for detecting a target substance alone while preventing nonspecific adsorption has been required.

As a technique for reducing nonspecific adsorption onto the substrate surface, a technique for reducing nonspecific adsorption of proteins by using oligo(ethylene glycol) methacrylate (OEGMA) as a monomer to form an OEGMA polymer on the surface plasmon resonance (SPR) sensor with high density by atom transfer radical polymerization is known. Furthermore, Japanese Patent Application Laid-Open No. 2009-57549 describes a technique for further preventing nonspecific adsorption by forming a crosslinked structure on the substrate surface. In this publication, however, techniques such as immobilizing a target substance-capturing body have not been described.

Meanwhile, Japanese Patent No. 2815120 describes a technique for detecting a target substance by forming a biocompatible porous matrix on the surface of a biosensor and further immobilizing a target substance-capturing body to a carboxyl group existing on the matrix to reduce nonspecific adsorption of impurities.

### DISCLOSURE OF THE INVENTION

Japanese Patent Application Laid-Open No. 2009-57549 reports a method for forming a membrane for reducing nonspecific adsorption of biomolecules onto the substrate surface and an effect of the membrane of reducing nonspecific adsorption, but dose not mention a method for immobilizing a target substance-capturing body on the membrane.

On the other hand, Japanese Patent No. 2815120 reports a method for reducing nonspecific adsorption of biomolecules onto the biosensor surface and then immobilizing a target substance-capturing body to detect the target substance alone. However, the effect of reducing nonspecific adsorption is not as high as that reported in Japanese Patent Application Laid-Open No. 2009-57549.

The present invention was accomplished based on such background art and provides a graft polymer-containing substrate for reducing nonspecific adsorption of impurities as well as specifically capturing a target substance by forming a polymer membrane on a substrate and immobilizing a target substance-capturing body that captures the target substance thereon, and a method for producing the same.

Furthermore, the present invention provides a target substance-detecting element and a target substance detection kit that detect a target substance alone with high sensitivity by using the above-mentioned graft polymer-containing substrate.

A graft polymer-containing substrate which solves the above-mentioned problem is a substrate with a graft polymer formed on the surface thereof, characterized in that the graft polymer includes monomer units represented by the following general formulas (1) and (2):

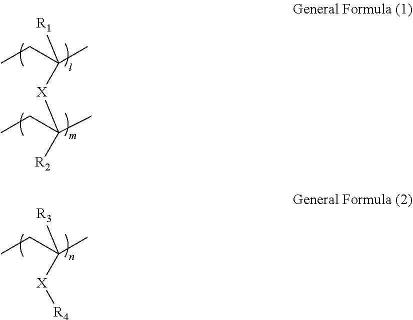

wherein R₁, R₂, and R₃ represent one of a hydrogen atom and a methyl group, R₄ represents a group including any of a carboxyl group, a succinimide group, an aldehyde group, an epoxy group, a bromo group, an amino group, and a maleimide group, X represents a hydrophilic crosslinking spacer having 15 or more atoms, and l, m, and n are an integer of 1 or greater (this definition is included in general formulas (1) and (2)).

A method for producing a graft polymer-containing substrate which solves the above-mentioned problem is a method for producing a graft polymer-containing substrate with a graft polymer formed on a surface thereof, characterized by including graft-polymerizing monomers represented by the following general formula (3) or (4) and introducing a functional group that is any of a carboxyl group, an aldehyde group, a succinimide group, an amino group, a maleimide group, and a glycidyl group by modifying an unreacted end of the above-mentioned monomer:

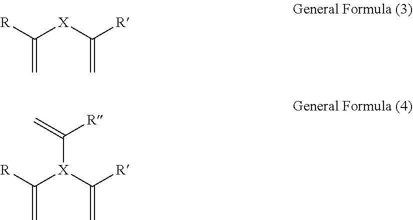

wherein R, R′, and R″ represent one of a hydrogen atom and a methyl group, and X represents a hydrophilic crosslinking spacer having 15 or more atoms (this definition is included in general formulas (3) and (4)).

A target substance-detecting element that solves the above-mentioned problem is characterized by including a substrate having a detection region, a graft polymer existing on the surface the substrate, and a first target substance-capturing body that binds to the polymer, the graft polymer including monomer units represented by general formulas (1) and (2) and the first target substance-capturing body binding to at least a part of an unreacted monomer end of the graft polymer:

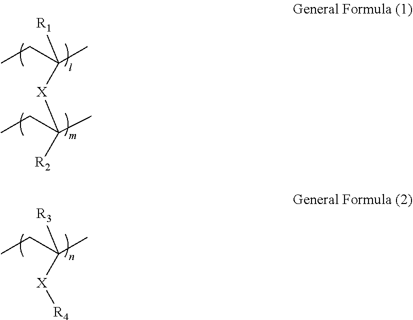

The graft polymer-containing substrate of the present invention can specifically capture a target substance by reducing nonspecific adsorption of impurities by a polymer membrane including a graft polymer bound with a target substance-capturing body. Furthermore, a target substance can be detected with high sensitivity by using a target substance-detecting element or a target substance detection kit with the above-mentioned graft polymer-containing substrate positioned in the detection region of the substrate.

Furthermore, the method for producing a graft polymer-containing substrate of the present invention can easily produce the above-mentioned graft polymer-containing substrate.

Further features of the present invention will become apparent from the following description of exemplary embodiments with reference to the attached drawings.

### BRIEF DESCRIPTION OF THE DRAWINGS

FIG. 1 is a schematic view illustrating one example of the graft polymer-containing substrate of the present invention.

FIG. 2 illustrates one example of the relationship between the graft density of the graft polymer and the crosslinking spacer length in the present invention.

FIG. 3 illustrates one example of the relationship between the graft density of the graft polymer and the X atom chain length in the present invention.

FIG. 4 illustrates one example of the relationship between the graft density of graft polymer and the PEG chain length in one example of the present invention.

FIG. 5 is a schematic view illustrating one example of the target substance-detecting element of the present invention.

FIG. 6 is a schematic view illustrating one example of the target substance detection kit of the present invention.

FIG. 7 illustrates IR-RAS analysis results in one example of the present invention.

### BEST MODES FOR CARRYING OUT THE INVENTION

Preferred embodiments of the present invention will now be described in detail in accordance with the accompanying drawings.

To solve the above-mentioned problem, the inventors of the present invention prepared a highly crosslinked graft polymer by grafting multivinyl monomers on a substrate with high density and found a graft polymer-containing substrate in which an unreacted vinyl group existing in the graft polymer was modified to a functional group for immobilization. According to the present invention, not only nonspecific adsorption of biomolecules onto the substrate surface can be reduced, but also a target substance-capturing body can be immobilized on the above-mentioned substrate surface.

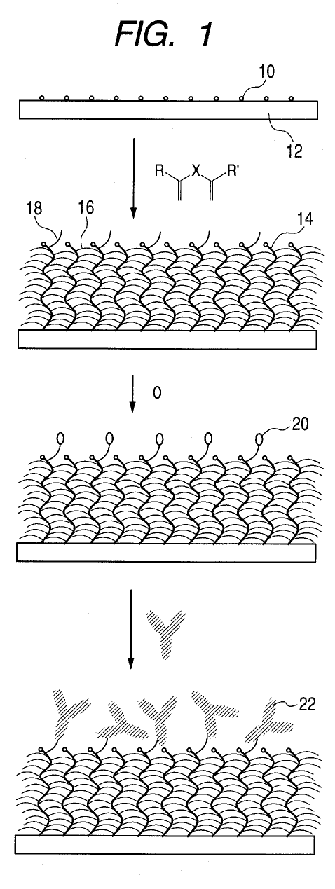

The construction of the graft polymer-containing substrate of the present invention will be described with reference to FIG. 1. FIG. 1 illustrates one example of the graft polymer-containing substrate of the present invention. A substrate 12 with a polymerization initiator 10 immobilized on the surface thereof is added to a solution containing multivinyl monomer molecules (divinyl monomer in FIG. 1) to allow polymerization. The graft polymer forms a crosslinked structure by polymerization of a plurality of vinyl groups in the multivinyl monomer. At this time, a main chain 14 of the graft polymer is extended by carbon-carbon bonds generally perpendicularly to the surface of the substrate 12. The X portion of the multivinyl monomer serves as a crosslinking spacer 16. In many cases, however, some multivinyl monomers have an uncrosslinked group particularly at an extension end. Specifically, some of vinyl groups in the graft polymer molecule are polymerized, and the other ends are free as uncrosslinked monomers 18. The free end of an uncrosslinked monomer is designated as a remaining vinyl group. A required molecule can be immobilized by modifying the remaining vinyl group to a functional group for immobilization 20. For example, a molecule such as a target substance-capturing body 22 can be immobilized.

The density of molecules immobilized onto the graft polymer varies depending on the size of each molecule to be immobilized or the amount of the remaining vinyl groups. Regarding the amount of the remaining vinyl groups, the remaining vinyl groups can be changed depending on amounts of multivinyl monomer molecules to be used and the reaction conditions when the graft polymer is formed. Therefore, according to the present invention, the density of molecules to be immobilized can be freely changed.

For example, when a large molecule such as an antibody is immobilized as a target substance-capturing body, the antibody molecule binds at many sites if many functional groups exist, resulting in reduction in the original activity of the antibody. To prevent reduction in the antibody activity, it is recommended to minimize the number of functional groups for immobilization. To this end, it is preferable that many of monomers are crosslinked in a graft polymer, and functional groups for immobilization exist with an interval which is larger than the size of an antibody molecule.

Furthermore, to reduce nonspecific adsorption, a higher percentage of crosslinking is preferred. Since a three-dimensional structure is formed by crosslinking, nonspecific adsorption is easily reduced as compared with a common polymer brush obtained by the substrate initiated polymerization. It has been demonstrated that a crosslinked graft polymer has a higher ability of reducing nonspecific adsorption than those of other graft polymers and coating materials. In the present invention, a target substance-capturing body can be immobilized by applying such a graft polymer.

The graft polymer-containing substrate of the present invention is a substrate with a graft polymer formed on the surface thereof, characterized in that the graft polymer includes monomer units represented by general formulas (1) and (2). It is preferable that a target substance-capturing body binds to at least a part of R₄.

The method for producing graft polymer-containing substrate of the present invention is a method for producing a graft polymer formed on a substrate surface, characterized by including graft-polymerizing a monomer represented by general formula (3) or (4) and introducing a functional group that is any of a carboxyl group, an aldehyde group, a succinimide group, an amino group, a maleimide group, and a glycidyl group by modifying an unreacted end of the monomer. In general formula 3 and 4, X is preferably an ethylene glycol polymer having four or more units.

The above-mentioned graft polymerization is more preferably living polymerization.

The above-mentioned living polymerization is yet more preferably living radical polymerization.

Hereafter, the graft polymer-containing substrate of the present invention will be described in detail.

### (Substrate)

Examples of the substrate used in the present invention include, but are not limited to, metals, metal oxides, silicon, glass, and plastics. The substrate can be of any material so long as a polymerization initiator for living polymerization can be immobilized, and the substrate is hardly affected by a chemical substance or a reaction solvent used for modifying a vinyl group. Any substrate can have a surface with reduced nonspecific adsorption of proteins by forming the graft polymer of the present invention on the surface.

The method for immobilizing a polymerization initiator on the substrate surface is not particularly limited. For example, when the substrate is a metal, a method is preferably employed in which a polymerization initiator containing a thiol compound is bound to the substrate surface, or the substrate is pretreated with a thiol compound before binding a polymerization initiator.

When the substrate surface is a metal oxide, silicon, or glass, a polymerization initiator containing a silane coupling agent can be bound. Alternatively, a method is preferably employed in which the above-mentioned substrate pretreated with a silane coupling agent before binding a polymerization initiator.

When the substrate is a plastic, a method is preferably employed in which the surface is oxidized by oxygen plasma treatment or UV treatment to express a carboxyl group before binding a polymerization initiator containing an amino compound, or the substrate is pretreated with an amino compound before binding a polymerization initiator.

Furthermore, the surface of the substrate of the present invention may be flat or curved. However, the graft density described later is based on a flat substrate. Therefore, when a curved substrate surface is formed, it is recommended to calculate the distance between main chains of the graft polymer taking a curvature factor into account. Application of a curved substrate will be described later.

### (Multivinyl Monomer)

The monomer used in the present invention is a compound represented by general formula (3) or (4).

Specifically, compounds having a plurality of a vinyl group, an isopropenyl group, an allyl group, an acryl group, and a methacryl group with carbon-carbon double bonds are used as the multivinyl monomer of the present invention. Compounds with a crosslinking spacer X having two or three carbon-carbon double bonds are shown above, but the crosslinking spacer X having four or more carbon-carbon double bonds can also be used as the multivinyl monomer of the present invention.

In the present invention, the length between two vinyl groups represented by the general formula (3) or (4) in a molecule is assumed as a crosslinking spacer length, and the number of atoms existing tandemly between the two vinyl groups is assumed as the atom chain length. The crosslinking spacer length is preferably a length exceeding an average length between main chains of the graft polymer. The main chain of the graft polymer of the present invention is a succession of carbon-carbon bonds and forms a chain extended generally perpendicularly to the substrate. Furthermore, when a multivinyl monomer having three or more carbon-carbon double bonds represented by the general formula (4) in the molecule is used, the atom chain length of any combination thereof preferably exceeds the distance between the main chains of the graft polymer.

### (Crosslinking Spacer Length)

The average length between main chains of the graft polymer is obtained from graft density and often varies greatly depending on the polymerization method. Therefore, the relationship between the graft density and the crosslinking spacer length is defined in the present invention. The range of the crosslinking spacer length is defined as follows.

When the distance between the main chains of the graft polymer is assumed as 2r (nm), and the area occupied by one main chain of the graft polymer is assumed as πr², the graft density is D=(πr)⁻¹ (chains/nm²). At this time, it is important that the crosslinking spacer length of the multivinyl monomer to be used, SL_{(X+2)} (nm), is greater than 2r. Specifically, when the crosslinking spacer length is within the range of

SL_{(X+2)}≦2r=2×(πD)^{-1/2} Equation (1)

the multivinyl monomer can be polymerized while maintaining the degree of freedom of the graft polymer main chain. When polymerization is performed under the above-mentioned condition, the degree of crosslinking of the graft polymer membrane constructed on the substrate increases. As a result, the graft polymer membrane can have a high size-exclusion characteristic. Furthermore, an effect of decreasing remaining vinyl groups after reaction can be obtained. Furthermore, when the concentration of the multivinyl monomer at polymerization is decreased as much as possible, an effect of further decreasing remaining vinyl groups can be obtained. Since hydrophilicity is improved by decrease in polymerization groups, this method is preferred as a method for reducing nonspecific adsorption to the above-mentioned prior art.

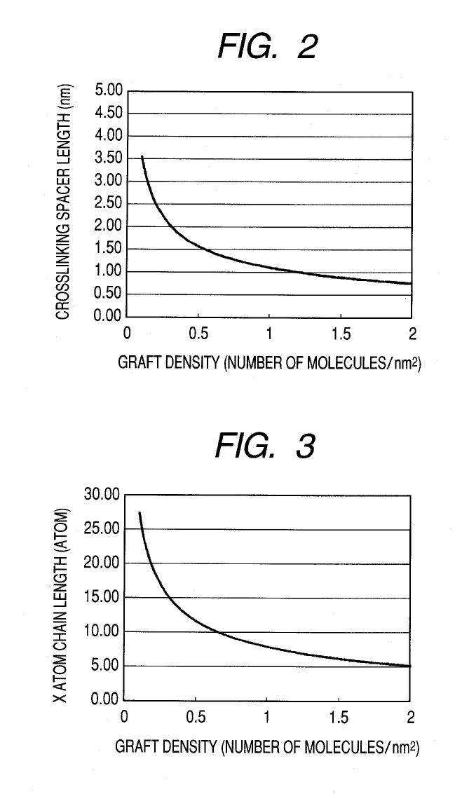

Furthermore, the curve in FIG. 2 illustrates the lower limits of the crosslinking spacer length SL_{(X+2)} based on graft density D in the present invention obtained by the equation 1. That is, in the present invention, a multivinyl monomer having a crosslinking spacer length above the curve in FIG. 2 is preferably used.

Preferably, a crosslinking spacer length of 0.3 nm or longer than the curve in FIG. 2 is used. When a multivinyl monomer is longer than the length between main chains of the graft polymer by about two atoms, interference by a reaction due to free rotation of a molecule chain is reduced, resulting in an increased reaction efficiency. More preferably, a crosslinking spacer length which is 0.5 nm or more longer than the curve in FIG. 2 is used. Accordingly, the degree of freedom of the whole graft polymer is increased, resulting in further improvement of the ability of reducing nonspecific adsorption.

Furthermore, when the substrate is curved, the crosslinking spacer length varies depending on the thickness of the graft polymer membrane. For example, the substrate is made of microparticles, and the radius of a microparticle including the graft polymer is (1+r) times the radius of the substrate microparticle (r represents the radius of the substrate microparticle), the required crosslinking spacer length is (1+r)² times longer than SL_{(X+2)} shown in FIG. 2. For example, when a graft polymer having a membrane thickness of 10 nm is produced on the microparticle surface with a microparticle having a diameter of 200 nm, a crosslinking spacer length 21% longer than SL_{(X+2)} in FIG. 2 is preferably used.

### (Atom Chain Length of Multivinyl Monomer)

When the range of the crosslinking spacer lengths in FIG. 2 is applied to the atom chain length of a multivinyl monomer, the atom chain length is expressed as follows.

First, the distance between atoms is considered. When the average length between bound atoms in the crosslinking spacer is assumed as B_{AVE}, the following equation is obtained for the X atom chain length CL_{X}(atom):

CL_{X}≦2/B_{AVE}×(πD)^{-1/2}−1 Equation (2)

The standard lengths between bound atoms are 0.154 nm with a C—C single bond, 0.149 nm with a C—N single bond, and 0.143 nm with a C—O single bond. Further, since the standard bond angle of the above-mentioned bond is 109.28°, and an effective length is obtained by multiplying each binding length by 0.816 (=cos [(180−109.28)/2]) (effective digits, 3 digits, same below). If a crosslinking spacer includes repeated C—C single bonds, the X atom chain length is obtained by assigning 0.126 (=0.154×0.816) to B in the equation 2, and the lower limits of the X atom chain length CL_{X} for the graft density D are expressed as the curve in FIG. 3. That is, a multivinyl monomer having an atom chain length in the range above the curve in FIG. 3 is preferably used.

In recent years, the graft density achieved by living polymerization using many monovinyl monomers is approx. 0.5 chains/nm². When it is assumed that the graft density of a monovinyl monomer is also applied to a multivinyl monomer, multivinyl monomers having X with a chain length of 15 atoms are applicable according to the equation 2 or FIG. 3. When the chain length of X is 15 or more atoms, binding of unreacted vinyl groups is considered to be hardly restricted because the crosslinking spacer length is longer than the distance between main chains of the graft polymer. Therefore, each multivinyl monomer is easily crosslinked, and remaining vinyl groups are reduced. In this case, the number of remaining vinyl groups corresponding to immobilized molecules is appropriate, and the remaining vinyl groups can be adjusted to a required number depending on reaction conditions. As a result, the ability of reducing nonspecific adsorption is increased, and the immobilized molecules easily function.

On the other hand, when a multivinyl monomer having X whose chain length is much shorter than 15 atoms (for example, X<11) is used, a phenomenon different from the above-mentioned phenomenon occurs. Specifically, it is considered that, when a vinyl group at one end is bound, a vinyl group at the other end and a main chain of the graft polymer are hardly brought into contact with each other because a crosslinking spacer length is short. While the above-mentioned vinyl group at the other end cannot react, and the other vinyl group binds to another multivinyl monomer. Therefore, the vinyl group at the other end cannot be bound and thus remains uncrosslinked. Therefore, each multivinyl monomer is hardly crosslinked, and the number of remaining vinyl groups increases. In this case, since the remaining vinyl groups corresponding to immobilized molecules are too many, many molecules are immobilized, but this is often unsuitable for specifications in view of many nonspecific adsorptions.

Furthermore, a multivinyl monomer having X whose chain length is close to 15 atoms (for example, X=12) is used, it is considered that a phenomenon totally different from the above-mentioned two phenomena occurs as a very rare case. It is considered that, since the crosslinking spacer length is virtually equal to the distance between main chains of the graft polymer, a vinyl group at one end is bound, and then a vinyl group at the other end immediately binds to an adjacent main chain of the graft polymer. A graft polymer extends rapidly, and the percentage of crosslinking is increased, but it is expected that the degrees of freedom of the graft polymer main chains and the whole graft polymer are deteriorated. Therefore, each multivinyl monomer is very easily crosslinked, and a very small number of remaining vinyl groups remain, but the ability of reducing nonspecific adsorption is considered to be low. However, when polymerization is performed at a high multivinyl monomer concentration to immobilize molecules, the number of remaining vinyl groups slightly increases. As a result, it is considered that the graft polymer maintains a degree of freedom, and the ability of reducing nonspecific adsorption increases. Furthermore, since molecules are moderately immobilized, the immobilized molecules easily function.

The effective atom chain length for the graft density obtained by recent techniques has been described above. When the graft density is improved by advanced technology, a monomer having X with a shorter atom chain length can also be used.

More preferably, an X atom chain length that is two or more atoms longer than the curve in FIG. 3 is applied. Specifically, in recent living polymerization, the degree of freedom of the whole graft polymer is increased by using a multivinyl monomer having X with a chain length of 15 or more atoms, and the ability of reducing nonspecific adsorption is further improved. Furthermore, the size of a mesh of the crosslinked structure is excessively larger than molecules of which nonspecific adsorption needs to be reduced, adsorption may occur. Therefore, it is considered that use of a multivinyl monomer having X with too long an atom chain length is not suitable for the present invention.

The curve in FIG. 3 illustrates the lower limits for a crosslinking spacer with successive C—C single bonds. When a crosslinking spacer has C—N single bonds or C—O single bonds, the lower limit is increased depending on the number of bonds. Furthermore, when bonds less than the above-mentioned bonds are included, hydrophilicity is reduced. Many bonds longer than the above-mentioned three single bonds exist and may have hydrophilicity. Examples thereof include P—O single bonds and S—O single bonds. In the case where such bonds are included, the lower limit of the X atom chain length can be determined taking into account the bond distance and the bond angle.

In the above description, the atom chain length of the multivinyl monomer of the present invention has been defined. However, after the graft polymer is constructed, the side chain may not be cleaved, or the main chain may also be cleaved if the side chain is cleaved. In this case, the distance between main chains of the graft polymer can also be predicted from the graft density of monovinyl monomers used in the grafting method as previously reported. If data about the graft density of monovinyl monomers having a certain side chain length (Y) is available, it can be expected that the graft density of multivinyl monomers having approx. twice the length of X atom chain length (2Y) is similar to the above-mentioned data.

### (Hydrophilic Multivinyl Monomer)

In the present invention, since a graft polymer obtained by polymerizing multivinyl monomers is hydrophilic, X of the above-mentioned multivinyl monomer has a hydrophilic element. X represents a hydrophilic crosslinking spacer. Oxygen atoms or nitrogen atoms are preferably used for X.

Hydrophilicity of X is described in detail below.

The representative example of X is an ethylene glycol polymer (PEG), which is highly hydrophilic and most commonly used. When the structural formula of a PEG is considered, B_{AVE}=(0.154×1+0.143×2)/3×0.816=0.120 is established. By assigning this value to the equation 2, the X atom chain length CLₓ when a multivinyl monomer having PEG as a side chain is used is obtained by equation 3.

CL_{X}≦16.7×(πD)^{-1/2}−1 Equation (3)

Furthermore, when the PEG unit number is assumed as UL_{PEG} (unit),

UL_{PEG}≦5.56×(πD)^{-1/2}−0.667 Equation (4)

is obtained from CL_{X}=3UL_{PEG}+1.

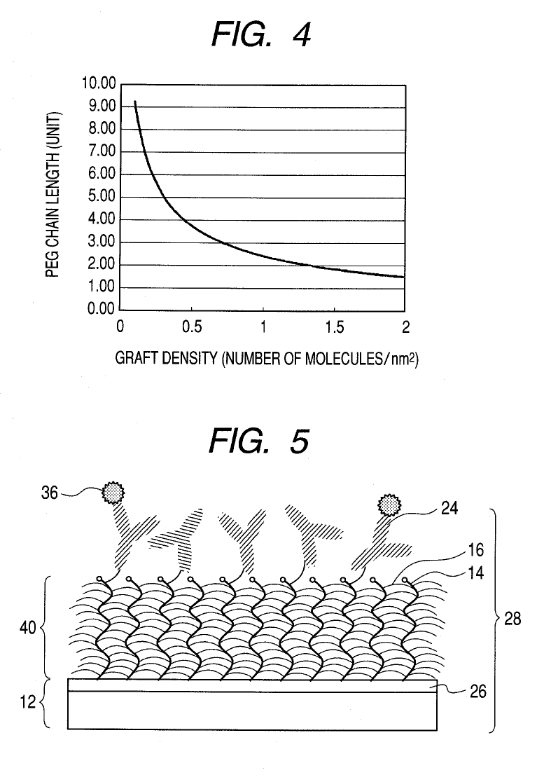

Furthermore, the curve in FIG. 4 is lower limits of the crosslinking spacer length UL_{PEG} for the graft density D in the present invention obtained from the equation 4. That is, a multivinyl monomer with a PEG unit number in the range above the curve in FIG. 4 is used.

However, FIG. 4 is applicable only to the cases where a vinyl group or an isopropenyl group is directly ether-bound to both ends of a PEG. When a multivinyl monomer includes bonds other than those to PEG (ester, amide, etc.) in X, the lower limit is obtained by adding lengths of other bonds.

Examples of PEG-containing multivinyl monomers that can be used in the present invention include, but are not limited to, polyethylene glycol divinyl ether, polyethylene glycol diallyl ether, polyethylene glycol diisopropenyl ether, polyethylene glycol diacrylate, and polyethylene glycol dimethacrylate.

Preferably, a PEG of four or more units is used. Here, the term “unit” is used to express the degree of polymerization of ethylene glycol. When a PEG of four or more units is used, a vinyl group at one end is reacted, and the degree of freedom of unreacted vinyl groups is hardly restricted due to a long side chain, resulting in further improvement of the reaction efficiency of unreacted vinyl groups. Furthermore, when a PEG of four or more units is used, the product easily becomes hydrophilic after graft polymerization considering the ratio of the graft polymer main chains and the side chains.

Furthermore, monomers having PEG in a part of X can also be used as multivinyl monomers. Examples thereof include, but are not limited to, monomers including in X a copolymer of ethylene glycol and propylene glycol, a copolymer of ethylene glycol and tetramethylene glycol, a copolymer of ethylene glycol and ethyleneimine, a copolymer of ethylene glycol and propyleneimine, a copolymer of ethylene glycol and tetramethyleneimine, a copolymer of ethylene glycol and glycerol, a copolymer of ethylene glycol and trimethylolpropane, a copolymer of ethylene glycol and pentaerythritol, a copolymer of ethylene glycol and triethanolamine, a copolymer of ethylene glycol and tris(2-aminoethyl)amine, and derivatives of the above-mentioned compounds.

Similarly, monomers having an ethyleneimine polymer in X can also be used as multivinyl monomers. Examples thereof include, but are not limited to, monomers including an ethyleneimine polymer, a copolymer of ethyleneimine and ethylene glycol, a copolymer of ethyleneimine and propylene glycol, a copolymer of ethyleneimine and tetramethylene glycol, a copolymer of ethyleneimine and propyleneimine, a copolymer of ethyleneimine and tetramethyleneimine, a copolymer of ethyleneimine and glycerol, a copolymer of ethyleneimine and trimethylol propane, a copolymer of ethyleneimine and pentaerythritol, a copolymer of ethyleneimine and triethanolamine, a copolymer of ethyleneimine and tris(2-aminoethyl)amine, and derivatives of the above-mentioned compounds.

Further, multivinyl monomers including a cation or an anion in X can also be used.

By using a multivinyl monomer including a cation, adsorption of molecules having a cation on the surface can be reduced by electric charge repulsion. Furthermore, when a multivinyl monomer including an anion is used, adsorption of molecules having an anion on the surface thereof can be similarly reduced.

Examples of multivinyl monomers including a cation include, but are not limited to, compounds including an amine and compounds including a quaternary ammonium ion.

Furthermore, examples of multivinyl monomers including an anion include, but are limited to, containing compounds including a carboxyl ion, compounds including a phosphate ion, and compounds including a sulfite ion.

Multivinyl monomers including an ampholytic ion pair in X can also be used.

When a multivinyl monomer has a cation and an anion in the molecule, nonspecific adsorption can be efficiently reduced. It is already known that betaine-containing compounds and phosphorylcholine-containing compounds are produced with monomethacrylate monomers by living radical polymerization and have an effect of reducing nonspecific adsorption.

Examples of multivinyl monomers including ampholytic ions include, but are not limited to, betaine-containing compounds, phosphorylcholine-containing compounds, and compounds obtained by linking amino acids. Betaine is a generic name for compounds which have a positive electric charge and a negative electric charge at positions not adjacent to each other in one molecule, and an atom having a positive electric charge not bound with a hydrogen atom that can dissociate (compounds having a cationic structure such as quaternary ammonium, sulfonium, and phosphonium), and have no electric charge as a whole molecule (intramolecular salts).

Furthermore, multivinyl monomers generated by an acid base reaction, such as a methacryloyl polyethylene glycol acid phosphate diethylaminoethyl methacrylate half salt obtained by mixing acid phosphooxy polyethylene glycol monomethacrylate and diethylaminoethyl monomethacrylate, can also used in the present invention.

Of the above-mentioned compounds, a graft polymer may be formed by polymerizing a mixture of a plurality of multivinyl monomers. Furthermore, a graft polymer may be formed by mixing the above-mentioned multivinyl monomers and a required monovinyl monomer.

### (Hydrophilicity)

The graft polymer of the present invention has hydrophilicity expressed by the contact angle of the graft polymer surface to water of 60° or less, preferably 40° or less.

Hydrophilicity for each unit was previously defined as follows. Hydrophilic compounds have six or more hydrogen bond acceptors (HBA), five or more hydrogen bond donors (HBD), or a total of nine HBA and HBD per molecule of the spacer. Furthermore, compounds that meet two or all of these conditions may be used. Nine or more HBA and six or more HBD are preferred (refer to WO2004/025297).

Here, the number of hydrogen bond acceptors (number of HBA) means a total number of included nitrogen atoms (N) and oxygen atoms (O) combined, and the number of hydrogen bond donors (number of HBD) means a total number of NH and OH combined (refer to Lipinski C. A. et al., Advanced Drug Delivery Reviews 23 (1997) 3-25).

In the present invention, the crosslinking spacer X of a multivinyl monomer represented by the general formula (1) or (2) may have a chain length of 15 or more atoms, and meet the above-mentioned conditions of the number of HBA and the number of HBD.

### (Crosslinked Structure)

When living polymerization is performed using multivinyl monomers, a side chain of the multivinyl monomer immediately serves as a spacer, and a crosslinked structure is spontaneously constructed. This is because a growing end of the polymer always has a polymerization activity (living) in living polymerization, and polymers with a matching chain length can be obtained. However, the degree of crosslinking changes greatly depending on the monomer chain length and the graft density. The degree of crosslinking is clarified by obtaining the ratio of remaining vinyl groups to all vinyl groups in a polymerized multivinyl monomers. The proportion of remaining vinyl groups can be obtained with a spectrophotometric technique described later. The graft polymer of the present invention preferably has 15% or less of remaining vinyl groups. Since the surface reaction activity becomes high if 15% or more of remaining vinyl groups are included, biomolecules and labeling substances are nonspecifically adsorbed onto the substrate surface.

More preferably, 2% or more and 15% or less of remaining vinyl groups are included. If less than 2% of remaining vinyl groups are included, the distance between main chains of the graft polymer and the crosslinking spacer length of a multivinyl monomer are considered to be similar. The graft polymer extends rapidly, and the degree of crosslinking increases, but degrees of freedom of the main chains of the graft polymer and the whole graft polymer are considered to be deteriorated. As a result, biomolecules and labeling substances may be nonspecifically adsorbed onto the substrate surface. If 2% or more and 15% or less of remaining vinyl groups are included, the graft polymer is considered to have a higher ability of reducing nonspecific adsorption.

The thickness of the graft polymer membrane is not limited so long as the thickness is within a reasonable range as the thickness of a membrane obtained by binding a polymer from the substrate surface. A thickness of 0.5 nm or greater is preferred. If the thickness is less than 0.5 nm, the size exclusion effect cannot be obtained, and nonspecific adsorption is likely to occur. Or, a graft polymer is absent depending on the size of a polymerization initiator.

The membrane thickness is more preferably 0.5 nm or greater and 10 nm or less. A membrane constructed by the graft polymer of the present invention has a crosslinked structure in many parts of the membrane. That is, since the membrane has a three-dimensional crosslinked structure, even a very thin membrane has a size exclusion effect, as compared with a graft membrane of monovinyl monomers that extend only in the longitudinal direction against the substrate. This property is utilized in many biosensors as an advantage. For example, in a surface plasmon resonance (SPR) sensor, a localized surface plasmon resonance (LSPR) sensor, or a magnetic sensor, the element surface is the most sensitive region. However, it is essential to reduce nonspecific adsorption in such biosensors. Therefore, a membrane thickness of several tens nanometers to several hundreds nanometers is conventionally required. When the present invention is used, the loss at the region with high sensitivity can be minimized, and nonspecific adsorption can be efficiently reduced.

### (Graft Density)

The graft density of a usual polymer brush obtained using monovinyl monomers can be obtained by calculating the number of graft polymer main chains per square nanometer from the membrane thickness and the length of the most extended chain (refer to p. 2, The 56th Annual Meeting Proceedings of The Society of Polymer Science, Japan). The graft density of the crosslinking graft membrane of the present invention may be obtained by this method after cleaving the crosslinking spacer portion.

In the present invention, a high graft density is defined as a state that graft polymer main chains exist with a density of 0.1 chains/nm² or higher. With such a high graft density, a dense crosslinked structure can be formed by polymerizing many end vinyl groups of a multivinyl monomer shown below. Preferably, when the graft density is 0.3 chains/nm² or higher, a denser crosslinked structure that can physically remove many protein impurities can be formed. More preferably, when the graft density is 0.4 chains/nm² or higher, degrees of freedom of the graft polymer main chains and the hydrophilic spacer can be maintained even by using commercially available relatively inexpensive multivinyl monomers such as a short chain of tetraethylene glycol dimethacrylate. The ability of reducing nonspecific adsorption can be improved by increasing the degrees of freedom of the graft polymer main chains and the hydrophilic spacer.

High graft density can be achieved by living polymerization. Furthermore, the number of living polymerization initiating groups can be adjusted to achieve a target graft density.

Preferably, living radical polymerization is used. It is known that the graft density achieved by living radical polymerization using many monovinyl monomers is approx. 0.5 chains/nm².

The living radical polymerization method will be described in detail below.

### (Living Radical Polymerization)

In general, the molecular weight distribution of a synthesized polymer is small, and a high density polymer layer can be grafted on the substrate by living radical polymerization. Therefore, radical polymerization of an appropriate chain length of multivinyl monomers enables each vinyl group to be reacted with the initiation point on the substrate, resulting in the formation of a high-density crosslink membrane on the substrate. Examples of the living radical polymerization method include methods utilizing any of the following polymerizations:

(1) Atom transfer radical polymerization (ATRP) using an organic halide as an initiator and transition metal complex as a catalyst;

(2) Nitroxide-mediated polymerization (NMP) using a radical scavenger such as a nitroxide compound; and

(3) Photoinitiator polymerization using a radical scavenger such as dithiocarbamate.

In the present invention, a graft polymer-containing substrate may be produced by any of these methods, but it is preferred to utilize atom transfer radical polymerization because of easy control.

### (Atom Transfer Radical Polymerization)

When atom transfer radical polymerization is performed as living radical polymerization, organic halides represented by chemical formulas (1) to (3) or a halogenated sulfonyl compound shown in chemical formula (4) can be used as a polymerization initiator.

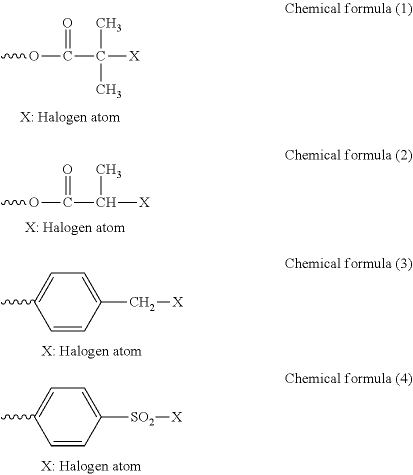

Atom transfer radical polymerization is performed by adding a substrate into which an atom transfer radical polymerization initiator is introduced to a reaction solvent, then adding multivinyl monomers and a transition metal complex, and replacing the atmosphere in the reaction system with an inert gas. By following these procedures, polymerization can be progressed while maintaining a constant graft density. That is, polymerization can be progressed in a living manner, and graft polymer chains are allowed to grow virtually uniformly on the substrate.

Reaction solvents are not particularly limited, and examples thereof include dimethyl sulfoxide, dimethylformamide, acetonitrile, pyridine, water, methanol, ethanol, propanol, butanol, pentanol, hexanol, heptanol, cyclohexanol, methyl cellosolve, ethyl cellosolve, isopropyl cellosolve, butyl cellosolve, acetone, methyl ethyl ketone, methyl isobutyl ketone, cyclohexanone, ethyl acetate, butyl acetate, ethyl propionate, trioxane, and tetrahydrofuran. Each of these may be used solely, or two or more may be used in combination.

As an inert gas, one of a nitrogen gas and an argon gas can be used.

The transition metal complex to be used includes a halogenated metal and a ligand. Transition metals from atom No. 22 (Ti) to No. 30 (Zn) are preferred as metal species of the halogenated metal, and Fe, Co, Ni, and Cu are more preferred. Of these, copper(I) chloride and copper(I) bromide are preferred.

The ligand is not particularly limited so long as the ligand can bind to a halogenated metal. Examples thereof include 2,2′-bipyridyl, 4,4′-di-(n-heptyl)-2,2′-bipyridyl, 2-(N-pentyliminomethyl)pyridine, (−)-sparteine, tris(2-dimethylaminoethyl)amine, ethylenediamine, dimethylglyoxime, 1,4,8,11-tetramethyl-1,4,8,11-tetraazacyclotetradecane, 1,10-phenanthroline, N,N,N′,N″,N″-pentamethyldiethylenetriamine, and hexamethyl(2-aminoethyl)amine.

The amount of a transition metal complex to be added is 0.001 to 10% by weight, preferably 0.05 to 5% by weight based on the multivinyl monomer.

The polymerization temperature is in the range of 10 to 100° C., preferably in the range 20 to 80° C.

After completion of polymerization, a graft polymer-containing substrate can be obtained by separating and purifying the substrate by an appropriate method such as filtration, decantation, fractionation by precipitation, or centrifugation.

### (Nitroxide-Mediated Polymerization)

When nitroxide-mediated polymerization is performed as living radical polymerization, nitroxide compounds represented by chemical formulas (5) to (7) can be used as a polymerization initiator.

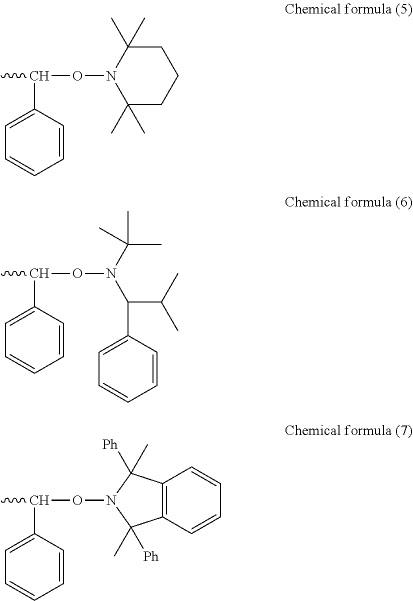

Nitroxide-mediated polymerization is performed by adding a substrate into which a nitroxide-mediated polymerization initiator is introduced to a reaction solvent, adding multivinyl monomers, and replacing the atmosphere in the reaction system with an inert gas. By following these procedures, polymerization can be progressed while maintaining a constant graft density. That is, living polymerization is progressed, and graft polymer chains are allowed to grow virtually uniformly on the substrate.

Reaction solvents are not particularly limited, but solvents similar to the above-mentioned solvents can be used. Furthermore, each of these may be used solely or two or more may be used in combination.

As an inert gas, one of a nitrogen gas and an argon gas can be used.

The polymerization temperature is in the range of 10 to 120° C., preferably in the range of 20 to 100° C. The polymerization temperature lower than 40° C. is not desirable because the formed graft polymer has a low molecular weight, or polymerization is hardly progressed.

After completion of polymerization, a graft polymer-containing substrate can be obtained by separating and purifying the substrate by an appropriate method such as filtration, decantation, fractionation by precipitation, or centrifugation.

### (Photoinitiator Polymerization)

When photoinitiator polymerization is performed as living radical polymerization, N,N-dithiocarbamate represented by chemical formula (8) can be used as a polymerization initiator.

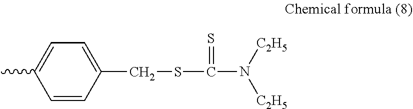

Photoinitiator polymerization is performed by adding a substrate into which a photoinitiator-polymerization initiator is introduced to a reaction solvent, adding multivinyl monomers, and replacing the atmosphere in the reaction system with an inert gas, and irradiating the substrate with light. By following these procedures, polymerization can be progressed while maintaining a constant graft density. That is, living polymerization is progressed, and graft polymer chains are allowed to grow virtually uniformly on the substrate.

Reaction solvents are not particularly limited, but similar solvents described above can be used. Furthermore, each of these may be used solely, two or more may be used in combination.

As an inert gas, one of a nitrogen gas and an argon gas can be used.

The wavelength of the applied light varies depending on the type of the photoinitiator-polymerization initiator to be used. When a polymer is grafted on the surface of a substrate having the photoinitiator-polymerization initiator represented by chemical formula (9), photoinitiator polymerization is favorably progressed by irradiating a reaction system with light having a wavelength of 300 to 600 nm.

The polymerization temperature is preferably room temperature or lower to prevent a side reaction. However, the temperature is not limited to this temperature region so long as similar effects can be obtained.

After completion of polymerization, a graft polymer-containing substrate can be obtained by separating and purifying the substrate by an appropriate method such as filtration, decantation, fractionation by precipitation, or centrifugation.

### (Modification of Vinyl Group)

A remaining vinyl group of a graft polymer obtained by polymerization is modified by a physical or chemical reaction without losing characteristics of the substrate.

To immobilize a target substance-capturing body, a remaining vinyl group can be modified to a functional group for immobilization, such as a carboxyl group, a succinimide group, an aldehyde group, an epoxy group, a bromo group, an amino group, or a maleimide group. Methods for modifying a remaining vinyl group to a functional group for immobilization are described below.

For example, a remaining vinyl group is modified to a carboxyl group by oxidation using potassium permanganate and an acid as shown in reaction formula (1).

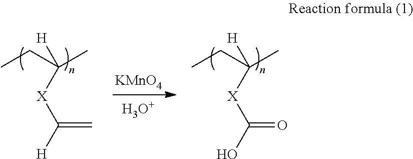

Furthermore, a remaining vinyl group is modified to a succinimide group by succinimidation of a carboxyl group by N-hydroxysulfosuccinimide (NHS) and/or 1-ethyl-3-(3-dimethylaminopropyl)carbodiimide (EDC) as shown in reaction formula (2).

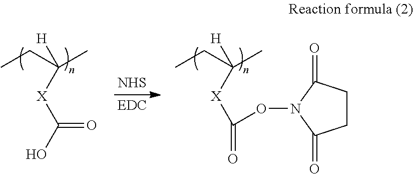

In methods other than the above-mentioned methods, a remaining vinyl group can be modified to a carboxyl group or a succinimide group. However, the above-mentioned reaction process is safe and easy. Since a succinimide group binds to an amino group of a target substance-capturing body and can immobilize the target substance-capturing body immediately, methods represented by the reaction formulas (1) and (2) are preferably used.

Furthermore, a remaining vinyl group is modified to an aldehyde group by ozone oxidation as shown in reaction formula (3).

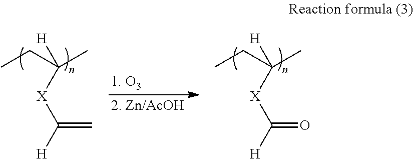

An aldehyde group binds to an amino group of a target substance-capturing body and can immobilize the target substance-capturing body immediately.

Furthermore, a remaining vinyl group is modified to an epoxy group by oxidation using peracid as shown in reaction formula (4).

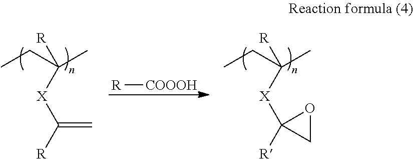

An epoxy group binds to a carboxyl group of a target substance-capturing body and can immobilize the target substance-capturing body immediately.

Furthermore, a remaining vinyl group is modified to a bromo group by Markovnikov addition using hydrogen bromide or anti-Markovnikov addition as shown in reaction formula (5) (reaction formula (5) represents anti-Markovnikov addition).

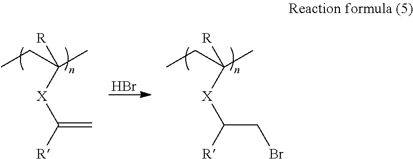

A bromo group binds to a hydroxyl group and a thiol group of a target substance-capturing body, and can immobilize the target substance-capturing body immediately.

Furthermore, a remaining vinyl group is modified to an amino group by substituting ethanolamine in a product of reaction formula (5) as shown in reaction formula (6).

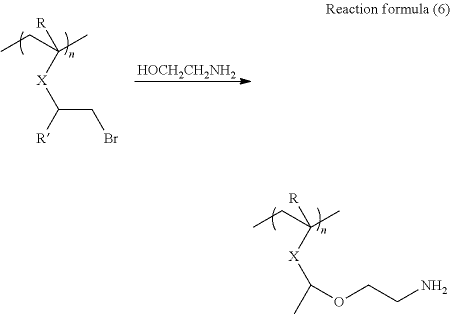

Furthermore, a remaining vinyl group is modified to a maleimide group by substituting ethanolamine in a product of reaction formula (6) as shown in reaction formula (7).

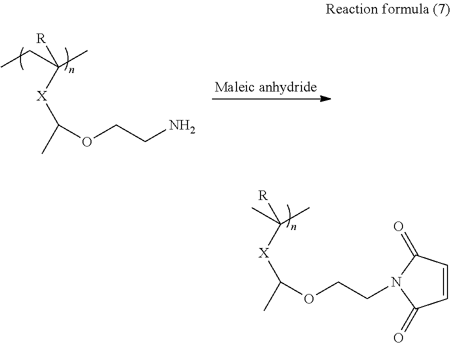

A maleimide group binds to a thiol group of a target substance-capturing body and can immobilize the target substance-capturing body immediately.

Methods for modifying a remaining vinyl group to a functional group for immobilization have been described above, but these methods are some examples of modification methods, and methods are not limited to the above methods.

### (Graft Polymer Evaluation Method)

In the present invention, the substrate surface is reformed by a graft polymer. Therefore, a graft polymer can be evaluated by analyzing the substrate surface, or analyzing products of a partially degraded graft polymer constructed on the substrate surface.

Hydrophilicity of a graft polymer can be evaluated by, for example, measuring a contact angle to water. The degree of crosslinking of a graft polymer and modification of a vinyl group can be evaluated by analyzing the proportion of unreacted C—C double bonds in reacted multivinyl monomers by methods such as infrared absorption (IR) spectroscopy and X-ray photoelectron spectroscopy (XPS). In particular, infrared reflection-absorption spectroscopy (IR-RAS) can analyze a thin film on a substrate with high sensitivity. IR-RAS is a technique by which orientation of a thin film is generally analyzed by detecting reflection of a polarized light. It has been found that a peak proportional to a membrane thickness of several nanometers to several tens nanometers is obtained with the graft polymer of the present invention. Therefore, this technique is useful as means for measuring remaining vinyl groups. For example, when a multivinyl monomer is acrylate or methacrylate, remaining vinyl groups can be quantified by using a peak derived from a carbonyl group as an internal standard.

Furthermore, the formation of a crosslinked structure can be evaluated by analyzing the relationship between strain and stress of a graft polymer with a rheometer.

The density of a graft polymer can be evaluated as follows, for example. The membrane thickness is measured by ellipsometry, and the substrate weight is measured before and after construction of a graft polymer. The crosslinking spacer is cleaved to separate the graft polymer from the substrate, and then the molecular weight of the graft polymer main chain is measured by gel permeation chromatography (GPC). Density can be evaluated using the membrane thickness and the molecular weight and the number of molecules of the graft polymer main chain obtained by the above measurement.

The length and the type of multivinyl monomers used for construction of a graft polymer can be evaluated by a combination of nuclear magnetic resonance (NMR) spectroscopy, infrared (IR) absorption spectroscopy, X-ray photoelectron spectroscopy (XPS), and time-of-flight secondary ion mass spectrometry (TOF-SIMS). Furthermore, the length and the type of multivinyl monomers can also be evaluated by using atomic force microscope (AFM) in combination.

The effect of reducing nonspecific adsorption of proteins to a graft polymer can be evaluated by fluorescence observation, fluorometry, enzyme-linked immunosorbent assay (ELISA), radioimmunoassay (RIA), SPR, LSPR, quartz crystal microbalance (QCM), and measurement using a magnetic sensor.

### (Target Substance-Detecting Element)

The target substance-detecting element of the present invention will be explained below.

The target substance-detecting element of the present invention is characterized by having a substrate including a detection region, a graft polymer on the surface of the substrate, a first target substance-capturing body that binds to the polymer, the graft polymer including a monomer unit represented by general formulas (1) and (2) and the first target substance-capturing body binding to at least a part of an unreacted monomer end of the graft polymer.

FIG. 5 is a schematic view illustrating the target substance-detecting element in the present invention.

In a target substance-detecting element 28, a substrate 12 has a detection region 26, and a graft polymer 40 is formed on the surface of the detection region 26. A first target substance-capturing body 24 (described later) is immobilized on the graft polymer 40, so that a target substance 36 (described later) can be captured.

The detection region 26 is a region for detecting the captured target substance. The detection region must be made of a material that transmits a signal derived from the captured target substance. For example, when surface plasmon resonance (SPR) or localized surface plasmon resonance (LSPR) is used as a detection method, a material that generates surface plasmon must be contained in the surface. When quartz crystal microbalance (QCM) is used as a detection method, a material whose frequency of resonance changes in proportion to the weight of the adsorbed substance must be contained.

When a field effect transistor (FET) is used in a detection method, a material that can pass an electric current and enables microfabrication must be contained. When a magnetic sensor is used in a detection method, a material whose magnetic pole is relatively easily eliminated or reversed, such as a soft magnetic substance, needs to be contained. When electrochemistry is used as the detection method, it is preferable to form a surface using a material having a potential region that does not inhibit an electrochemical reaction. When light absorption is used as the detection method, it is preferable to form a surface using a material that transmits light of a wavelength to be detected. When fluorescence or luminescence is used as a detection method, a surface is preferably made of a material that does not absorb a light of a wavelength to be detected.

Examples of the material of the detection region include, but are not limited to, gold, silver, copper, platinum, quartz, silicon, germanium, zinc oxide, titanium oxide, oxidation silicon, indium oxide, cadmium sulfide, cadmium selenide, gallium arsenide, permalloy (Ni—Fe alloy), Co—Fe—B alloy, mercury, carbon, diamond, glass, and plastic.

These materials of the detection region are often selected depending on the method for detecting a target substance. When SPR is used in a detection method, the material of the detection region is preferably a metal such as gold, silver, copper, or platinum. Gold is preferred. When QCM is used in a detection method, the material of the detection region is preferably quartz. When an FET is used in a detection method, examples of the material of the detection region include silicon, germanium, zinc oxide, titanium oxide, oxidation silicon, oxidation indium, cadmium sulfide, cadmium selenide, and gallium arsenide. As described above, when SPR, QCM, or FET is used in a detection method, a target substance can be detected without being labeled.

Furthermore, when a magnetic sensor is used in a detection method, the material of the detection region is preferably a soft magnetic substance represented by permalloy (Ni—Fe alloy) or Co—Fe—B alloy. When electrochemistry is used as a detection method, the material of the detection region is preferably gold, platinum, mercury, carbon, or diamond. When light absorption, fluorescence, or luminescence is used as a detection method, the material of the detection region is preferably glass or plastic.

The detection region 26 may exist on the surface of the substrate 12 or inside the substrate 12. For example, the detection region 26 exists on the surface of the substrate 12 when SPR or electrochemistry is used. When SPR is used, a metal thin film that generates surface plasmon must be formed on the surface of the substrate. The target substance-detecting element of the present invention can be prepared by forming a graft polymer on the metal thin film. When electrochemistry is used, a metal must be formed at least on the surface of an action electrode.

Meanwhile, the detection region 26 may exist inside the substrate 12, for example, when QCM, an FET, or a magnetic sensor is used. When QCM is used, the target substance-detecting element of the present invention can be prepared by forming a graft polymer on a gold thin film formed on a quartz surface. When an FET is used, the target substance-detecting element of the present invention can be prepared by forming a graft polymer on the surface of a structure made of a material that passes an electric current. When a magnetic sensor is used, the target substance-detecting element of the present invention can be prepared by forming a graft polymer on a layer formed on the surface of a plurality of layers containing permalloy (Ni—Fe alloy) or Co—Fe—B alloy.

FIG. 5 illustrates an example of the case where the surface of the substrate 12 is a detection region 26. The substrate 12 includes a plurality of layers, and a layer including the surface of the substrate 12 among these layers is a detection region 26. However, the substrate 12 is not limited to the above-described example, and may be a layer not including the surface among a plurality of layers or may have a structure where the substrate 12 is formed with one layer, and the whole substrate 12 is a detection region 26.

### (Target Substance Detection Kit)

The target substance detection kit of the present invention will be explained below.

The target substance detection kit of the present invention is characterized by including the above-mentioned target substance-detecting element and a labeling material including a labeling substance and a second target substance-capturing body existing on the surface of the labeling substance.

The above-mentioned detection region is a detection region that can detect a magnetic substance, and the above-mentioned labeling substance is preferably a magnetic substance.

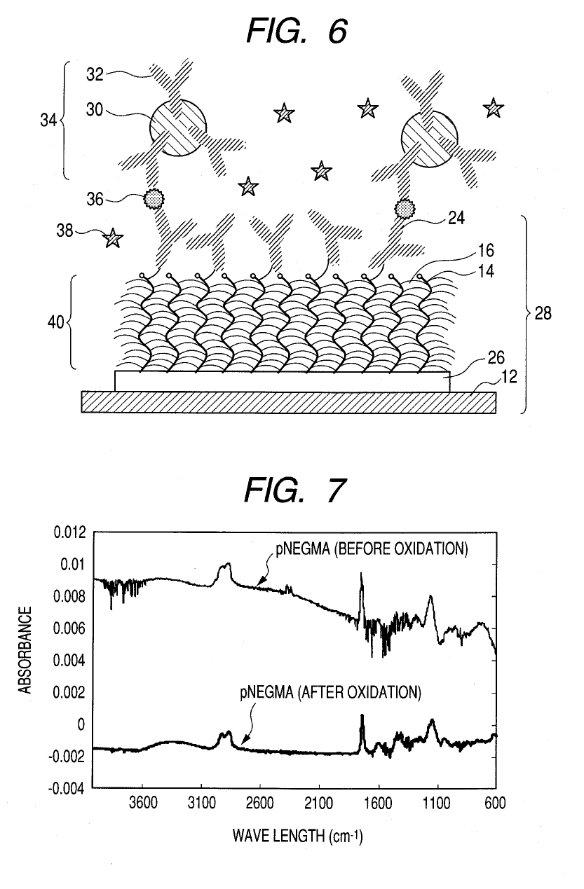

FIG. 6 is a schematic view illustrating the target substance detection kit of the present invention.

The target substance detection kit includes a target substance-detecting element 28 and a labeling material 34.

The target substance-detecting element 28 is similar to the above-mentioned target substance-detecting element, and the detection region 26 of the target substance-detecting element 28 is a detection region that can detect a labeling substance material of the labeling material 34.

The labeling material 34 includes a labeling substance 30 and a second target substance-capturing body 32 existing on the surface of the labeling substance.

Examples of the labeling substance 30 include gold colloid, latex beads, luminol, ruthenium, enzymes, radioactive substances, fluorescent substances, and magnetic substances. Specific examples of fluorescent substances include quantum dot, fluorescent proteins (for example, GFP and derivatives thereof), Cy3, Cy5, Texas Red, fluorescein, and Alexa dyes (for example, Alexa 568). Specific examples of enzymes include horseradish peroxidase, alkaline phosphatase, β-galactosidase, and luciferase. Examples of magnetic substances include ferrites. Ferrites are preferred because ferrites have a sufficient magnetic property under bioactive conditions, and deterioration such as oxidation is unlikely to occur in a solvent. A ferrite is selected from magnetite (Fe₃O₄), maghemite (γ-Fe₂O₃), and complexes in which a part of these Fe is substituted with another atom. Another atom is at least any of Li, Mg, Al, Si, Ca, Sc, Ti, V, Cr, Mn, Co, Ni, Cu, Zn, Ga, Ge, Zr, Nb, Mo, Cd, In, Sn, Ta, and W. Furthermore, the labeling substance may be a complex of the above-mentioned materials.

Examples of the shape of the labeling substance 30 include a particle shape, a columnar shape, and a star shape, and a particle shape is preferred. When the labeling substance 30 has a particle shape, the average grain size is preferably 1 nm to 100 μm, more preferably 3 nm to 10 μm. When the labeling substance has a particle shape, the average grain size can be measured by a dynamic light scattering method.

As a magnetic substance, for example, Dynabeads commercially available from Dynal, micromer-M and nanomag-D commercially available from Micromod, or Estapor commercially available from Merck can be used.

The second target substance-capturing body 32 is immobilized on the surface of the labeling substance 30 and has a function of capturing a target substance 36 as with the first target substance-capturing body 24 of the target substance-detecting element 28. Therefore, examples of the second target substance-capturing body 32 are similar to examples of the first target substance-capturing body 24. It is noted that the second target substance-capturing body 32 and the first target substance-capturing body 24 need to capture different parts of a target substance 36. In this case, a complex in the form of a target substance 36 sandwiched between the second target substance-capturing body 32 and the first target substance-capturing body 24 is formed. Furthermore, for example, when the second target substance-capturing body 32 and the first target substance-capturing body 24 are monoclonal antibodies, these antibodies must be of different types. However, when the second target substance-capturing body 32 and the first target substance are polyclonal antibodies, antibodies may be of the same type or of different types. Of two target substance-capturing bodies, one may be a monoclonal antibody, and the other may be a polyclonal antibody.

Specifically, a target substance 36 is captured by the first target substance-capturing body 24 positioned on the surface of the target substance-detecting element 28, and the target substance 36 captured by the first target substance-capturing body 24 is further captured by a second target substance-capturing body 32 of a labeling material 34. That is, a complex of first target substance-capturing body 24—target substance 36—second target substance-capturing body 32 is formed, and the labeling material 34 is positioned in the vicinity of the detection region 26 of the target substance-detecting element 28. The labeling substance 30 of the labeling material 34 positioned in the vicinity of the detection region 26 is detected by the detection region 26, and detection of the labeling substance 30 in the detection region 26 occurs as an electrical or physical signal change of the target substance-detecting element 28. The presence or absence or the number of a target substance is detected by utilizing this signal change of the target substance-detecting element 28.

Here, it is assumed that a target substance 36 is captured by the first target substance-capturing body 24, and then the target substance 36 is captured by the second target substance-capturing body 32. However, the target substance 36 may be captured by the second target substance-capturing body 32, and then the target substance 36 captured by the second target substance-capturing body 32 may be captured by the first target substance-capturing body 24.

Examples of such a combination of the target substance-detecting element 28 and the labeling substance 30 include the following. For example, when the target substance-detecting element 28 is a magnetic sensor element, the labeling substance 30 is a magnetic substance. When the target substance-detecting element 28 is an electrode, the labeling substance 30 is an enzyme. When the target substance-detecting element 28 is a microtiter plate, the labeling substance 30 is gold colloid, latex beads, luminol, ruthenium, an enzyme, a radioactive substance, or a fluorescent substance.

As described above, when the target substance-detecting element 28 is a magnetic sensor element, the magnetic sensor element can detect the presence or absence or the number of a magnetic substance positioned in the vicinity of the detection region 26. In such a case, the target substance-detecting element 28 has a detection region that detects a change in magnetic property, and, for example, a magnetic resistance effect element, a hall effect element, or a superconducting quantum interference element can be suitably used.

### (Target Substance and First Target Substance-Capturing Body)

It is sufficient that the first target substance-capturing body 24 is a molecule that captures or converts a target substance 36 by interacting with a target substance 36. Examples of such a first target substance-capturing body 24 include nucleic acids, proteins, sugar chains, lipids and complexes thereof. Specific examples thereof include, but are not limited to, DNA, RNA, aptamers, genes, chromosomes, cell membranes, viruses, antigens, antibodies, antibody fragments, lectins, haptens, hormones, receptors, enzymes, peptides, glycosphingos, and sphingo lipids. Preferred examples include antibodies, antibody fragments, or enzymes that capture or convert a biological substance.

Target substances 36 are not limited so long as the substances react with the first target substance-capturing body 24. Biological substances are more preferred. Biological substances include biological substances selected from nucleic acids, proteins, sugar chains, lipids, and complexes thereof, more specifically substances including a biomolecule selected from nucleic acids, proteins, sugar chains, and lipids. Specifically, the present invention can be applied to any substance so long as the substance includes any substance selected from DNA, RNA, aptamers, genes, chromosomes, cell membranes, viruses, antigens, antibodies, lectins, haptens, hormones, receptors, enzymes, peptides, glycosphingos, and sphingo lipids. Furthermore, bacteria or cells themselves producing the above-mentioned “biological substances” can also be a target substance as a target “biological substance” of the present invention.

Therefore, examples of these interactions of the target substance 36 and the first target substance-capturing body 24 include “antigen-antibody reactions,” “antigen-aptamer (RNA fragment having a specific structure),” “ligand-receptor interactions,” “DNA hybridizations,” “DNA-protein (transcription factor, etc.) interactions,” and “lectin-sugar chain interactions.” Examples of the interactions between the target substance 36 and the second target substance-capturing body 28 are similar.

Nonspecific adsorption of impurities 38 can be reduced by having the structures described above.

### EXAMPLES

Hereafter, the present invention will be more specifically described with reference to examples. However, the scope of the present invention is not limited to these examples, and materials, compositions, and reaction conditions can be modified so long as a magnetic biosensor having similar function or effect can be obtained.

### Example 1

### Step of Introducing ATRP Initiator onto Gold Thin Film Substrate

A gold thin film substrate of SIA Kit Au (GE Healthcare Bio-sciences) was subjected to ultrasonic cleaning successively in acetone, isopropyl alcohol, and ultrapure water. The gold thin film substrate was dried under a nitrogen purge, then loaded in UV/O₃ Washing Apparatus UV-1 (SAMCO, Inc.), and washed with UV/O₃ at 120° C. for 10 min. The washed gold thin film substrate was immersed in ultrapure water again to perform ultrasonic cleaning.

The washed gold substrate thin film was immersed in an ethanol solution containing an atom transfer radical polymerization (ATRP) initiator represented by chemical formula (9) to produce a gold thin film substrate with a Self Assembly Monolayer (SAM) formed thereon with the ATRP initiator. After the substrate was dried under a nitrogen purge, the thickness of the SAM membrane measured with Ellipsometer M-2000 (J. A. Woollam) was 1.86±0.08 nm.

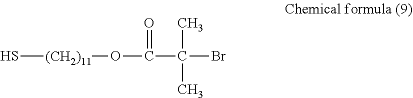

(Step of Constructing Graft Polymer on SAM Surface with ATRP Initiator)

A SAM-formed substrate was placed in a Schlenk flask for reaction and secured so that the substrate should not hit the wall surface. Subsequently, the Schlenk flask was placed in ice water, and 2,2′-bipyridyl, nonaethylene glycol diacrylate monomer represented by chemical formula (10) (NEGDA, Shin-Nakamura Chemical Co., Ltd., NK Ester A-400), and methanol/ultrapure water (4/1=w/w) were added. The atmosphere in the reaction system was replaced with nitrogen by charging nitrogen into the Schlenk flask with a syringe. Copper(I) bromide was added, followed by further nitrogen substitution, and ATRP was initiated at 23° C. After a reaction for 24 h, the reaction was terminated by exposure to the atmosphere.

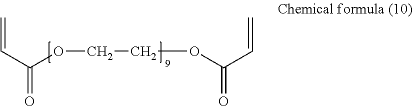

After termination of the reaction, the substrate was washed with methanol and ultrapure water to obtain a graft polymer-containing substrate crosslinked with NEGDA. After the substrate was dried under a nitrogen purge, the thickness of the graft polymer membrane measured as described above was 2.3±0.1 nm (excluding the SAM membrane thickness). Furthermore, the contact angle to water was 25±2°.

### (Step of Oxidizing Graft Polymer)

Acetic acid and a permanganate potassium solution were added to methanol containing 18-crown-6 dissolved therein, and it was confirmed that the whole solution had changed to purple-red. The obtained NEGDA graft polymer-containing substrate was immersed in the solution, and the solution was stirred overnight. The substrate was washed with methanol to obtain an oxidized graft polymer. After the substrate was dried under a nitrogen purge, the thickness of the graft polymer membrane measured as described above was 2.1±0.1 nm (excluding the SAM membrane thickness). Furthermore, the contact angle to water was 12±2°. FIG. 7 illustrates the results of IR-RAS analyses before and after oxidation of the surface of graft polymer-containing substrate. Since the analysis results after oxidation show that a peak in the vicinity of 1300 cm⁻¹ derived from a vinyl group disappeared, and a peak in the vicinity of 3300 cm⁻¹ derived from a carboxyl group appeared, it is demonstrated that the graft polymer has been oxidized.

### (Evaluation of Functions of Oxidized Graft Polymer)

Quantities of antigen-antibody reactions and nonspecific binding adsorptions were measured with Biacore X based on SPR as a principle (GE Healthcare Bio-sciences). A sensor chip was prepared according to a method described in the manual attached to SIA Kit Au using the oxidized NEGDA graft polymer and inserted into Biacore X by a specified method. The substrate surface and the passage were washed with a phosphate buffer (pH 7.4) by a specified method, and sensorgram was initiated at a flow rate of 5 μl/min. A mixed aqueous solution of N-hydroxysulfosuccinimide (NHS) and 1-ethyl-3-(3-dimethylaminopropyl)carbodiimide (EDC) was allowed to flow to activate the graft polymer surface. Then, an anti-human chorionic gonadotropin antibody (Anti-hCG, Medix Biochemica) solution was allowed to flow, and the antibody was immobilized on the graft polymer surface at 659 RU. Further, an ethanolamine solution was allowed to flow to inactivate active groups. The flow rate was changed to 20 μl/min, and then a 1 μg/ml of a human chorionic gonadotropin antigen (hCG, Rohto Pharmaceutical Co., Ltd.) solution was allowed to flow for 2 min. The difference in the signals at 5 min after completing the flow of the protein solutions and before allowing the solutions to flow was obtained as the quantity of reaction of hCG and found to be 105 RU. When the remaining activity of the immobilized antibody was estimated, it was confirmed that 48% of the immobilized antibody had captured the antigen.

Furthermore, to check nonspecific adsorption at an impurity protein concentration of a serum level, a 1% bovine immunoglobulin G (BIgG) solution was allowed to flow for 2 min instead of the above-mentioned hCG solution. The difference of signals at 5 min after completing the flow of the protein solution and before allowing the protein solution to flow was obtained as the quantity of nonspecific adsorption of BIgG and found to be 37 RU.

### Example 2

### Step of Introducing ATRP Initiator onto Gold Thin Film Substrate

A gold thin film substrate of SIA Kit Au (GE Healthcare Bio-sciences) was subjected to ultrasonic cleaning successively in acetone, isopropyl alcohol, and ultrapure water. The gold thin film substrate was dried under a nitrogen purge, then loaded in UV/O₃ Washing Apparatus UV-1 (SAMCO, Inc.), and washed with UV/O₃ at 20° C. for 10 min. The washed gold thin film substrate was immersed in ultrapure water again to perform ultrasonic cleaning.

The washed gold substrate thin film was immersed in an ethanol solution containing an atom transfer radical polymerization (ATRP) initiator represented by chemical formula (9) to produce a gold thin film substrate with a self assembly monolayer (SAM) formed thereon with the ATRP initiator. After the substrate was dried under a nitrogen purge, the thickness of the SAM membrane measured with Ellipsometer M-2000 (J.A. Woollam) was 1.86±0.08 nm.

(Step of Constructing Graft Polymer on SAM Surface with ATRP Initiator)

A SAM-formed substrate was placed in a Schlenk flask for reaction and secured so that the substrate should not hit the wall surface. Subsequently, the Schlenk flask was placed in ice water, and 2,2′-bipyridyl, an ethoxylated glycerine triacrylate monomer represented by chemical formula (11) (Shin-Nakamura Chemical Co., Ltd., A-GLY-20E), and methanol/ultrapure water (4/1=w/w) were added. The atmosphere in the reaction system was replaced with nitrogen by charging nitrogen into the Schlenk flask with a syringe. Copper(I) bromide was added, followed by further nitrogen substitution, and ATRP was initiated at 23° C. After a reaction for 24 h, the reaction was terminated by exposure to the atmosphere.

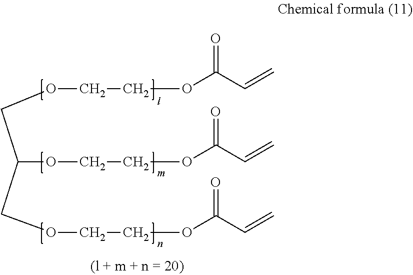

Alter termination or the reaction, the substrate was washed with methanol and ultrapure water to obtain a graft polymer-containing substrate crosslinked with a monomer represented by chemical formula (11). After the substrate was dried under a nitrogen purge, the thickness of the graft polymer membrane measured as described above was 1.9±0.2 nm (excluding the SAM membrane thickness). Furthermore, the contact angle to water was 29±2°.

### (Step of Oxidizing Graft Polymer)

Acetic acid and a permanganate potassium solution were added to methanol containing 18-crown-6 dissolved therein, and it was confirmed that the whole solution had changed to purple-red. The obtained graft polymer-containing substrate was immersed in the solution, and the solution was stirred overnight. The substrate was washed with methanol to obtain an oxidized graft polymer. After the substrate was dried under a nitrogen purge, the thickness of graft polymer membrane measured as described above was 1.7±0.2 nm (excluding the SAM membrane thickness). Furthermore, the contact angle to water was 15±3°. IR-RAS analyses were performed before and after oxidation of the surface of graft polymer-containing substrate. Since the analysis results after oxidation show that a peak in the vicinity of 1300 cm⁻¹ derived from a vinyl group disappeared, and a peak in the vicinity of 3300 cm⁻¹ derived from a carboxyl group appeared, it is demonstrated that the graft polymer has been oxidized.

### (Evaluation of Functions of Oxidized Graft Polymer)

Quantities of antigen-antibody reactions and nonspecific binding adsorptions were measured with Biacore X based on SPR as a principle (GE Healthcare Bio-sciences). A sensor chip was prepared according to a method described in the manual attached to SIA Kit Au using the oxidized graft polymer and inserted into Biacore X by a specified method. The substrate surface and the passage were washed with a phosphate buffer (pH 7.4) by a specified method, and sensorgram was initiated at a flow rate of 5 μl/min. A mixed aqueous solution of N-hydroxysulfosuccinimide (NHS) and 1-ethyl-3-(3-dimethylaminopropyl)carbodiimide (EDC) was allowed to flow to activate the graft polymer surface. Then, an anti-human chorionic gonadotropin antibody (Anti-hCG, Medix Biochemica) solution was allowed to flow, and the antibody was immobilized on the graft polymer surface at 805 RU. Further, an ethanolamine solution was allowed to flow to inactivate active groups. The flow rate was changed to 20 μl/min, and then a 1 μg/ml of human chorionic gonadotropin antigen (hCG, Rohto Pharmaceutical Co., Ltd.) solution was allowed to flow for 2 min. The difference in the signals at 5 min after completing the flow of the protein solutions and before allowing the solutions to flow was obtained as the quantity of reaction of hCG and found to be 202 RU. When the remaining activity of the immobilized antibody was estimated, it was confirmed that 75% of the immobilized antibody had captured the antigen.

Furthermore, to check nonspecific adsorption at an impurity protein concentration of a serum level, a 1% bovine immunoglobulin G (BIgG) solution was allowed to flow for 2 min instead of the above-mentioned hCG solution. The difference of signals at 5 min after completing the flow of the protein solution and before allowing the protein solution to flow was obtained as the quantity of nonspecific adsorption of BIgG and found to be 10 RU.

### Comparative Example 1

Sensor Chip CM5 (GE Healthcare Bio-sciences) was inserted into Biacore X by a specified method. The substrate surface and the passage were washed with a phosphate buffer (pH 7.4) by a specified method, and sensorgram was initiated at a flow rate of 5 μl/min. A mixed solution of an NHS solution and an EDC solution was allowed to flow to activate the chip surface. Then, an anti-hCG solution was allowed to flow, and the antibody was immobilized on the chip surface at 498 RU. Further, an ethanolamine solution was allowed to flow to inactivate active groups. The flow rate was changed to 20 μl/min, and then a 1 μg/ml of hCG solution was allowed to flow for 2 min. The difference in the signals at 5 min after completing the flow of the protein solutions and before allowing the solutions to flow was obtained as the quantity of reaction of hCG and found to be 54 RU. When the remaining activity of the immobilized antibody was estimated, it was confirmed that 32% of the immobilized antibody had captured the antigen.

Furthermore, to check nonspecific adsorption at an impurity protein concentration of a serum level, a 1% bovine immunoglobulin G (BIgG) solution was allowed to flow for 2 min instead of the above-mentioned hCG solution. The difference of signals at 5 min after completing the flow of the protein solution and before allowing the protein solution to flow was obtained as the quantity of nonspecific adsorption of BIgG and found to be 197 RU.

### Example 3

This is an example in which a magnetic biosensor with a polymer membrane containing a primary antibody that captures prostate-specific antigen (PSA) formed in a detection region as a first target substance-capturing body and a magnetic marker including magnetite provided with a secondary antibody that captures PSA as a second target substance-capturing body is prepared, and PSA is detected as a magnetic biosensor. To detect a magnetic biosensor, a magnetic resistance effect element is used.

### (Preparation of Magnetic Marker)

First, a magnetic marker having a secondary antibody that captures PSA as a second target substance-capturing body is prepared.

Magnetite particles (average grain size, 100 nm) are heat-treated under a dry nitrogen atmosphere and dispersed in anhydrous toluene. To this magnetite particle/toluene dispersion is added aminopropyltrimethoxysilane, a silane coupling agent, to introduce an amino group into a magnetite particle surface. Then, to immobilize a secondary antibody that captures PSA as a second target substance-capturing body, the above-mentioned amino group and an amino group of the secondary antibody covalently bound using a glutaraldehyde crosslinking agent, so that the second target substance-capturing body can be immobilized onto the magnetite particle surface.

By following the above procedures, a magnetic marker having the second target substance-capturing body can be obtained.

### (Preparation of Magnetic Biosensor)

Subsequently, a magnetic biosensor with a polymer membrane having a primary antibody that captures PSA as a first target substance-capturing body in the detection region is prepared.

First, as shown in FIG. 6, an Au membrane is formed on the upper surface of a detection region of a magnetic biosensor. In this example, since a magnetic resistance effect element is used in the detection method, the detection region means a magnetic resistance effect membrane.

Subsequently, a polymer membrane is formed on the Au surface, which serves as the detection region. First, the Au membrane is immersed in an ethanol solution containing an atom transfer radical polymerization initiator represented by chemical formula (9) to react the above-mentioned initiator and the Au membrane, so that the atom transfer radical polymerization initiator can be introduced into the Au membrane surface.

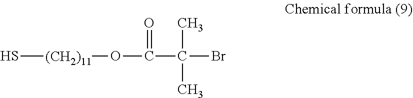

Subsequently, the detection region into which the atom transfer radical polymerization initiator is introduced is immersed in a water-methanol mixed solvent (volume ratio, 4:1), and CuBr and 2,2′-bipyridyl are added. Oxygen is removed from the reaction system by vacuum freeze-drying deaeration and then replaced with nitrogen, and NEGDA monomers represented by chemical formula (12) are reacted by atom transfer radical polymerization for a predetermined period. After polymerization, the substrate is washed with methanol and then with water to obtain a NEGDA graft polymer (polymer of chemical formula (12) on the detection region.

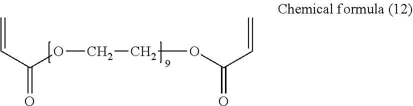

Subsequently, acetic acid and a permanganate potassium solution are added to methanol containing 18-crown-6 dissolved therein, and it is confirmed that the whole solution has changed to purple-red. The detection region is immersed in the solution, and the solution is stirred overnight. The substrate is washed with methanol to obtain a detection region with an oxidized graft polymer.

Subsequently, a primary antibody that captures PSA is immobilized to a side chain carboxyl group of the polymer membrane as the first target substance-capturing body. First, the substrate is similarly coated with a mixed aqueous solution of NHS and EDC. By following these procedures, a succinimide group (active ester group) is exposed to the side chain carboxyl group of the polymer membrane. The above-mentioned succinimide group and an amino group of the primary antibody are reacted to immobilize the primary antibody that captures PSA as the first target substance-capturing body. Then, unreacted succinimide groups on the polymer membrane are inactivated with ethanolamine.

By following the above procedures, a magnetic biosensor having in the detection region a polymer membrane on which a primary antibody captures PSA as the first target substance-capturing body is immobilized can be prepared.

### (Detection of Psa)

By following the procedures below using the magnetic marker and the magnetic biosensor prepared by the above-mentioned procedures, detection of PSA known as a marker of prostatic cancer can be attempted.

1) The detection region of the above-mentioned magnetic biosensor is immersed in a phosphate buffer containing PSA, a target substance (antigen), BSA, an impurity, and IgG.

2) Unreacted PSA and impurities are washed with a phosphate buffer.

3) The detection region of the above-mentioned magnetic biosensor after being subjected to Steps 1) and 2) is immersed in a phosphate-buffered physiological saline containing magnetic markers and incubated for 5 min.

4) Unreacted magnetic markers are washed with a phosphate buffer.

By following the above-mentioned procedures, the target substance (antigen) is captured by the first target substance-capturing body and the second target substance-capturing body, and the magnetic marker is immobilized on the detection region of the magnetic biosensor as shown in FIG. 6. That is, if no antigen exists in a specimen, the magnetic marker is not immobilized on the detection region of the magnetic biosensor. Therefore, the antigen can be detected by detecting the presence or absence of a magnetic marker. Furthermore, the amount of the antigen contained in the specimen can be indirectly determined by examining the number of immobilized magnetic markers.

The crosslinked graft polymer in the polymer membrane of the detection region of the magnetic biosensor in this example reduces nonspecific adsorption of impurities and the target substance (antigen) contained in the specimen, minimizing a noise, so that the target substance can be detected with high sensitivity.

### INDUSTRIAL APPLICABILITY

The graft polymer-containing substrate of the present invention is very useful as a biosensor material because it has an excellent ability of reducing nonspecific adsorption of biomolecules and labeling substances onto a substrate surface and can immobilize a target substance-capturing body.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-143457, filed May 30, 2008, which is hereby incorporated by reference in its entirety.

1. A graft polymer-containing substrate with a graft polymer formed on a surface thereof, wherein the graft polymer includes monomer units represented by the following general formulas (1) and (2): 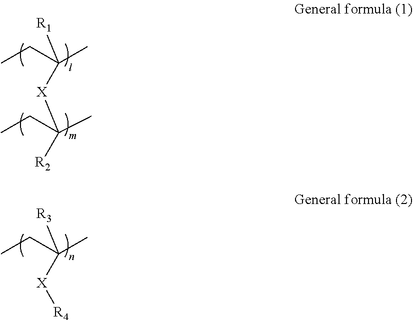
wherein R₁, R₂, and R₃ represent one of a hydrogen atom and a methyl group, R₄ represents a group including any of a carboxyl group, a succinimide group, an aldehyde group, an epoxy group, a bromo group, an amino group, and a maleimide group, X represents a hydrophilic cros slinking spacer having 15 or more atoms, and l, m, and n are an integer of 1 or greater.

2. The graft polymer-containing substrate according to claim 1, wherein X has an ethylene glycol polymer of four or more units.

3. The graft polymer-containing substrate according to claim 1, wherein a target substance-capturing body binds to at least a part of the R₄.

4. A method for producing a graft polymer-containing substrate with a graft polymer formed on a surface, comprising graft-polymerizing a monomer represented by the following general formula (3) or (4) and introducing a functional group of any of a carboxyl group, an aldehyde group, a succinimide group, an amino group, a maleimide group, and a glycidyl group by modifying an unreacted end of the monomer: 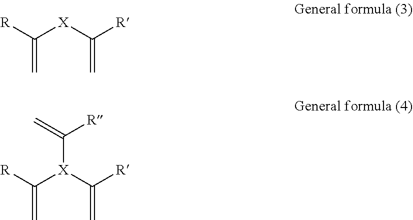
wherein R, R′, and R″ represent one of a hydrogen atom and a methyl group, and X represents a hydrophilic crosslinking spacer having 15 or more atoms.

5. The method for producing a graft polymer-containing substrate according to claim 4, wherein the monomer represented by the general formula (3) or (4) is graft-polymerized on the substrate surface with a polymerization initiator immobilized thereon.

6. The method for producing a graft polymer-containing substrate according to claim 4, wherein the X represents an ethylene glycol polymer of four or more units.

7. The method for producing a graft polymer-containing substrate according to claim 4, wherein the graft polymerization is living polymerization.

8. The method for producing a graft polymer-containing substrate according to claim 4, wherein the living polymerization is living radical polymerization.

9. A target substance-detecting element, comprising a substrate having a detection region, a graft polymer existing on the surface of the substrate, and a first target substance-capturing body binding to the polymer, the graft polymer including monomer units represented by the general formulas (1) and (2) and the first target substance-capturing body binding to at least a part of an unreacted end of a monomer of the graft polymer: 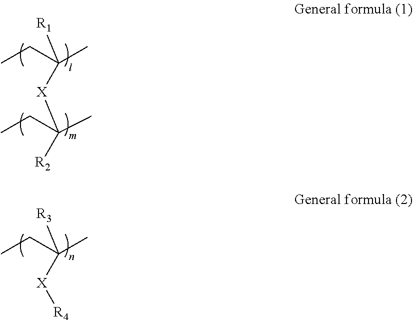
wherein R₁, R₂, and R₃ represent one of a hydrogen atom and a methyl group, R₄ represents a group including any of a carboxyl group, a succinimide group, an aldehyde group, an epoxy group, a bromo group, an amino group, and a maleimide group, X represents a hydrophilic cros slinking spacer having 15 or more atoms, and l, m, and n are an integer of 1 or greater.

10. A target substance detection kit, consisting of the target substance-detecting element according to claim 9 and a labeling material comprising a labeling substance and a second target substance-capturing body existing on the surface of the labeling substance.

11. The target substance detection kit according to claim 10, wherein the detection region can detect a magnetic substance, and the labeling substance is a magnetic substance.
